(19) United States
(12) Patent Application Publication
(10) Pub. No.: US 2023/0190824 A1
(43) Pub. Date: Jun. 22, 2023

(54) BACTERIUM OF THE CHRISTENSENELLACEAE FAMILY IN THE PREVENTION AND/OR TREATMENT OF HYPERTRIGLYCERIDEMIA

(71) Applicant: YSOPIA BIOSCIENCES, BORDEAUX CEDEX (FR)

(72) Inventors: Georges RAWADI, PARIS (FR); Sandrine, Paule CLAUS, BORDEAUX (FR); Laure RINALDI, BORDEAUX (FR); Frederic ELUSTONDO, BORDEAUX (FR); Marion, Sophie, Madeleine SOTO, MERIGNAC (FR); Katy LECORF, MERIGNAC (FR)

(21) Appl. No.: 17/912,190

(22) PCT Filed: Apr. 24, 2020
(86) PCT No.: PCT/EP2020/061510

§ 371 Date: Sep. 16, 2022

Related U.S. Application Data
(60) Provisional application No. 62/839,215, filed on Apr. 26, 2019.

(30) Foreign Application Priority Data
Apr. 25, 2019 (FR) FR1904346

(51) Int. Cl. A61K 35/74; A61K 35/741; A61K 45/06; A61K 9/19; A61P 3/06

(52) U.S. Cl. CPC A61K 35/74; A61K 9/19; A61K 35/741; A61K 45/06; A61P 3/06; A61K 2035/11

(57) ABSTRACT

The invention relates to a bacterium of the Christensenellaceae family or of a composition containing same for use in the prevention and/or treatment of hypertriglyceridemia in humans or animals.

The invention relates to the treatment of hypertriglyceridemia using specific bacteria of the gut microbiota.

Triglycerides are lipids that allow the storage of fatty acids in adipose tissue. They can be hydrolyzed as needed to allow the release of fatty acids that are then used as a source of energy.

Hypertriglyceridemia corresponds to an excess of circulating triglycerides, i.e. contained in the blood. In particular, hypertriglyceridemia corresponds to an increase in circulating triglyceride levels above 150 mg/dL, i.e. 1.7 mmol/L according to the guidelines of the NCEP ATP III, American Heart Association, National Lipid Association, the Endocrine Society, the European Society of Cardiology and the European Atherosclerosis Society.

The disease can be classified into 2 categories:
primary hypertriglyceridemia: of genetic origin
secondary hypertriglyceridemia: induced by drug treatments, excessive intake of alcohol, poor eating habits, diabetes, endocrine disease, kidney disease, liver disease, pregnancy or autoimmune disorders.

Although hypertriglyceridemia is a common metabolic disorder in obese individuals, obesity is not the only cause of hypertriglyceridemia. It is therefore appropriate to differentiate obesity as a disease associated with excess weight, and hypertriglyceridemia as a disease associated with an excess of circulating triglycerides, which makes them distinct pathological conditions.

The prevalence of hypertriglyceridemia is estimated at about 32% of the US population, including 18% above the limit of 200 mg/dL (moderate to severe hypertriglyceridemia).

Hypertriglyceridemia may cause complications and lead to the appearance of other pathologies. In fact, moderate hypertriglyceridemia is a major risk factor for the development of cardiovascular disease, such as atherosclerosis. It is also known that one of the main pathologies associated with severe hypertriglyceridemia is acute pancreatitis.

Currently, there are several types of treatments:
Modification of lifestyle and adoption of a diet rich in or supplemented with omega 3: this type of treatment is recommended for minor and moderate hypertriglyceridemia without associated risk. However, management with only a lifestyle change has a limited impact and usually leads to a progression of the disease to severe hyperlipidemia.
statins (lipid-lowering agents): they act mainly on elevated LDL-cholesterol but not on elevated triglycerides, which has nevertheless been attributed to an increased risk of vascular accidents. In addition, statins have significant side effects, mainly muscle pain. In some cases, they have been shown to cause elevated blood glucose and precipitate type 2 diabetes.
fibrates: they are recommended in the treatment of moderate to severe hypertriglyceridemia with a high risk of cardiovascular events. However, they are often associated with liver toxicity and are contraindicated in combination with many drugs, especially antibiotics.
niacin: it has limited effectiveness and significant harmful effects. It is limited to restricted use and is not recommended by US health authorities in combination with statins.

Thus, none of the present treatments are satisfactory, and there is a great need for an effective treatment of hypertriglyceridemia that is well tolerated and therefore can be administered in the long term without side effects, either alone or co-administered with another existing lipid-lowering drug to potentiate its effects and thus reduce the necessary dose and therefore the side effects.

This is the objective of the present invention, which, to respond to this need, focuses on the use of particular bacteria of the human gut microbiota, namely bacteria of the family Christensenellaceae.

Bacteria of the family Christensenellaceae, including the genus Christensenella, have already been studied and described. This is the case in particular for Christensenella minuta, Christensenella massiliensis and Christensenella timonensis. Christensenella minuta in particular was described for the first time in 2012. In 2014, a study showed that it was the most heritable taxon in a cohort of British twins and that its presence is associated with a low body mass index. This correlation between Christensenella minuta and low body mass index was then observed in a dozen studies published since 2014 in geographically diverse populations.

Surprisingly, and according to the invention, the bacteria of the family Christensenellaceae, in particular of the genus Christensenella, and in particular Christensenella minuta, when administered to humans or animals, are capable of decreasing circulating triglycerides and lipoproteins, such as LDL (low density lipoproteins) and VLDL (very low-density lipoproteins).

Therefore, the subject of the invention is a bacterium of the family Christensenellaceae, for its use in the prevention and/or treatment of hypertriglyceridemia in humans or animals.

Advantageously, such a bacterium, when administered to a human or an animal with hypertriglyceridemia, can act on excess triglycerides in the body and on lipoproteins that allow their transport.

The invention also relates to compositions comprising at least one bacterium of the family Christensenellaceae for its use in the prevention and/or treatment of hypertriglyceridemia in humans or animals.

Other features and advantages will become apparent from the detailed description of the invention which follows.

### FIGURES

FIG. 1 shows the quantification of triglycerides (FIG. 1A) and free fatty acids (FIG. 1B) of the liver of mice fed with a high-fat diet (HFD45%) having received a control solution (HFD-Veh) or a solution containing C. minuta 10⁹ CFU (HFD-C minuta) by daily gavage for 7 weeks.

FIG. 2 shows the level of relative expression of glucokinase (Gck) in the liver of mice fed with a high-fat diet (HFD45%) having received a control solution (HFD-Veh) or a solution containing C. minuta 10⁹ CFU (HFD-C minuta) by daily gavage for 4 weeks.

### DETAILED DESCRIPTION OF THE INVENTION

The invention therefore relates to the use, in the prevention and/or treatment of hypertriglyceridemia in humans or animals, of at least one bacterium of the family Christensenellaceae.

The invention therefore relates to a bacterium of the family Christensenellaceae for use in the prevention and/or treatment of hypertriglyceridemia, particularly in persons or animals with secondary hypertriglyceridemia. Preferably, the bacteria are chosen from Christensenella massiliensis, Christensenella timonensis and/or Christensenella minuta and mixtures thereof.

The invention also relates to a bacterium of the family Christensenellaceae for use in the prevention and/or treatment of primary hypertriglyceridemia in humans or animals. Preferably, the bacteria are chosen from Christensenella massiliensis, Christensenella timonensis and/or Christensenella minuta and mixtures thereof.

According to the invention, bacteria of the family Christensenellaceae, when administered to a human or animal with hypertriglyceridemia, are able to act on excess circulating triglycerides during hypertriglyceridemia, and on LDL and VLDL lipoproteins. These molecules are directly at the origin of the disease and their decrease can prevent and/or treat the pathology. In particular, the invention makes it possible to reduce the level of circulating triglycerides below the threshold of 150 mg/dL and that of LDL lipoproteins below the threshold of 1.6 g/L.

The useful bacterium or bacteria according to the invention are administered to humans or animals in an amount effective for an action on at least one of these molecules, i.e., to reduce the production of at least one of these molecules in the body. According to a suitable embodiment, the bacterium or bacteria can be administered at a dose of 10⁶ to 10¹² colony-forming units (CFU) per day, regardless of the weight of the person or animal. It will preferably be a single dose, i.e., administered once daily, or a dose before each meal (three times a day).

Preferentially, the bacterium of the family Christensenellaceae is also useful in the treatment of at least one disease caused by hypertriglyceridemia, such as atherosclerosis or acute pancreatitis.

The useful bacterium or bacteria according to the invention are bacteria of the family Christensenellaceae, preferably of the genus Christensenella. It may be, in particular, Christensenella massiliensis, Christensenella timonensis and/or Christensenella minuta and mixtures thereof. According to a particularly suitable variant, it is Christensenella minuta. According to another variant, it is Christensenella massiliensis and Christensenella timonensis or Christensenella massiliensis and Christensenella minuta or Christensenella timonensis and Christensenella minuta or Christensenella massiliensis and Christensenella timonensis and Christensenella minuta.

These bacteria can be isolated from human stools for example according to the protocols published by Morotomi et al., 2012 (Morotomi, M., Nagai, F. & Watanabe, Y. Description of Christensenella minuta gen. nov., sp. nov., isolated from human faeces, which forms a separate branch in the order Clostridiales, and proposal of Christensenellaceae fam nov. INTERNATIONAL JOURNAL OF SYSTEMATIC AND EVOLUTIONARY MICROBIOLOGY 62, 144-149 (2012)) and NDongo et al., 2016 (Ndongo, S., Dubourg, G., Khelaifia, S., Fournier, P. E. & Raoult, D. Christensenella timonensis, a new bacterial species isolated from the human gut. New Microbes and New Infections 13, 32-33 (2016)). These documents also describe the culture methods of the bacteria that are useful according to the invention.

The useful bacterium or bacteria according to the invention, for their previously described use, are preferably administered in a composition.

Thus, the subject of the invention is also a composition comprising at least one bacterium of the family Christensenellaceae for the prevention and/or treatment of hypertriglyceridemia in humans or animals. The subject of the invention is also a composition comprising at least one bacterium and/or at least one protein and/or at least one nucleic acid coming from at least one bacterium of the family Christensenellaceae for the prevention and/or treatment of hypertriglyceridemia in humans or animals. The bacterium or bacteria are present in an effective amount in the composition, allowing an effect on the hypertriglyceridemia of the treated persons or animals.

Preferably, the useful composition according to the invention comprises 10⁶ to 10¹² colony-forming units (CFU) of bacteria of the family Christensenellaceae per daily dose of composition to be administered. Preferably, this corresponds to a daily dose of bacteria to be administered, regardless of the weight of the person or the animal. Preferably, this dose is administered once per day.

The useful composition according to the invention may be in liquid form. It may in particular comprise bacteria of the Christensenellaceae family and a culture medium for said bacteria that makes it possible to preserve them, such as, for example, Columbia anaerobic medium enriched with sheep blood, or an equivalent medium not containing an animal byproduct.

According to one variant, the useful composition according to the invention may be in solid form. In this case, the bacteria may be present in freeze-dried form, and may also comprise excipients such as, for example, microcrystalline cellulose, lactose, sucrose, fructose, levulose, starches, stachyose, raffinose, amylum, calcium lactate, magnesium sulphate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, sorbitol and/or glycerol.

The useful compositions according to the invention may be in the form of powder, microencapsulated powder, gelcap, capsule, tablet, lozenge, granules, emulsion, suspension or suppository. According to a particularly suitable embodiment, they may be in a gastro-resistant form, such as a coated tablet containing microencapsulated bacteria.

The bacteria can be used alive, or inactivated, for example by heat, exposure to an appropriate pH, gamma radiation or high pressure.

They can all be alive or all inactivated.

Preferably, at least part of the bacteria is made up of live bacteria, in particular at least 50% (by number), even more preferably at least 90% (by number).

Thus, according to a suitable embodiment, the bacteria present in the useful composition according to the invention are at least 50% living bacteria (by number), preferably at least 90% living bacteria (by number), and even more preferentially all living.

The storage conditions according to the invention are for liquid formulations in the form of a frozen product maintained at -20° C. in a sealed bag. For solid formulations, the storage conditions according to the invention comprise a capsule or a coating hermetically sealed against light and oxygen, maintained at an ambient temperature of between 15° C. and 40° C. and a humidity level between 3% and 70%.

The useful bacteria according to the invention, and in particular the compositions that include it, can be administered orally, topically, inhaled or rectally.

The useful compositions according to the invention, in addition to the useful bacteria according to the invention, can comprise other compounds, such as:
at least one probiotic, and/or
at least one bacterium producing lactic acid, which makes it possible to create an anaerobic environment favorable to Christensenellaceae, such as at least one bacterium chosen from bacteria of the genus Lactobacillus spp., Bifidobacterium spp., Streptococcus spp. and/or at least one other organism promoting the anaerobic conditions necessary for the survival of Christensenellaceae, such as at least one yeast chosen from Saccharomyces spp. or microorganisms of the Methanobacteriaceae family, and/or
at least one bacterium associated with the Christensenellaceae ecosystem, since they facilitate their survival in the intestine, such as at least one bacterium chosen from bacteria of the phylum Firmicutes, Bacteroidetes, Actinobacteria, Tenericutes, and Verrucomicrobia, and/or
at least one bacterium chosen from bacteria of the order Clostridales, Verrucomicrobiales, Aeromonadales, Alteromonadales, ML615J-28, RF32, YS2, of the family Clostridiaceae, Lachnospiraceae, Ruminococcaceae, Bacteroidaceae, Enterococcaceae, Rikenellaceae, Dehalobacteriaceae, Veillonellaceae, and/or
at least one bacterium chosen from bacteria of the genus Faecalibacterium, Akkermansia, Eubacterium, Turicibacter and Oscillospira such as for example Faecalibacterium prausnitzii, Akkermansia muciniphila, Eubacterium halii, Turicibacter sanguinis, Oscillospira guilliermondii, and/or
at least one prebiotic such as for example at least one prebiotic chosen from galactooligosaccharides, fructooligosaccharides, inulins, arabinoxylans, beta-glucans, lactoglobulins and/or beta-caseins, and/or
at least one polyphenol such as for example at least one polyphenol chosen from quercetin, kaempferol, resveratrol, flavones (such as luteolin), flavan-3-ols (such as catechins), flavanones (such as naringenin), isoflavones, anthocyanidins, proanthocyanidins, and/or
at least one mineral and/or at least one vitamin and/or at least one nutritional agent, and/or
at least one pharmaceutical active ingredient preferably chosen from omega 3, statins, fibrates, niacin.

The invention is now illustrated by examples of useful bacteria according to the invention, methods of culturing these bacteria, examples of compositions containing them and test results demonstrating the effectiveness of the invention.

### EXAMPLES

### Example 1: Christensenella Minuta

Christensenella minuta can be cultivated according to the operating protocol described as follows.
1/ Dissolve a dehydrated RCM (“Reinforced Clostridial Medium”) medium in distilled water
2/ Add 0.5 mL/L of resazurin-Na solution (0.1 % w/v)
3/ Bring to a boil and cool to room temperature while injecting a gaseous mixture of 80% N₂ and 20% CO₂
4/ Spread the medium under the same gaseous atmosphere in anoxic Hungate-type tubes or in serum vials, then autoclave
5/ Before use, add 1.0 g of sodium carbonate per liter from a sterile anoxic stock solution prepared with a gaseous mixture of 80% N₂ and 20% CO₂
6/ Check the pH of the medium after autoclaving and adjust the pH between 7.3 and 7.5, using a sterile anoxic stock solution of sodium bicarbonate (5% w/v) prepared in a gaseous atmosphere at 80% N₂ and at 20% CO₂.

### Example 2: Christensenella Massiliensis

Christensenella massiliensis can be cultivated according to the operating protocol described as follows.

1/ Prepare a carboxymethylcellulose (N₂/CO₂) medium by following the instructions below provided by DSMZ (Deutsche Sammlung von Mikroorganismen und Zellkulturen).

2/ Dissolve the different constituents listed in the table below, except cysteine, carbohydrates and carbonate.

TABLE 1

| | |
|---|---|
| Casitone | 30.0 g |
| Yeast extract | 5.0 g |
| K2HPO₄ | 5.0 g |
| Na-resazurin solution (0.1 % w/v) | 0.5 mL |
| L-Cysteine-HCl x H₂O | 0.5 g |
| D-Glucose | 4.0 g |
| Cellobiose | 1.0 g |
| Maltose | 1.0 g |
| Starch (soluble) | 1.0 g |
| Na₂CO₃ | 1.5 g |
| Meat filtrate (see Table 2) | 1000 mL |

3/ Boil the medium for 1 min, then let it cool to room temperature under a gaseous atmosphere containing 80% N₂ and 20% CO₂.

4/ Add 0.5 g/L of L-cysteine-HCl x H₂O and pour it under the same gaseous atmosphere into Hungate-type tubes (for strains requiring meat particles, introduce these first into tube; use 1 part meat particles to 4 or 5 parts liquid).

5/ Autoclave at 121° C. for 20 min.

6/ After autoclaving, add glucose, cellobiose, maltose and starch from sterile anoxic stock solutions prepared with 100% N₂ gas and carbonate from a sterile anoxic stock solution prepared under gaseous mixtures at 80% N₂ and 20% CO₂.

7/ Adjust the pH of the medium to 7, if necessary.

### Preparation of the Meat Filtrate

TABLE 2

| | |
|---|---|
| Ground meat (no fat) | 500.0 g |
| 1 N NaOH | 25.0 mL |
| Distilled water | 1000 mL |

Use lean beef or horse meat.
Remove fat and connective tissue before chopping.
Mix the meat, water and NaOH, then boil for 15 minutes with stirring.
Allow to cool to room temperature, remove fat from the surface and filter, retaining meat particles and filtrate.
Add water to the filtrate to a final volume of 1000.0 mL.

The bacteria must be grown under anaerobic conditions at 37° C.

### Example 3: Christensenella Timonensis

Christensenella timonensis can be cultivated according to the same procedure as that described in Example 2 for Christensenella massiliensis.

### Example 4: Useful Composition According to the Invention in Liquid Form

An example of a useful composition according to the invention in liquid form is a composition comprising Christensenella minuta 10⁹ CFU/mL in the RCM anaerobic culture medium described above, modified to contain no product of animal origin and enriched with 5% glycerol.

The composition of Example 4 was obtained from an RCB (“research cell bank”) prepared with Christensenella minuta 10¹⁰ CFU/mL and stored frozen at -20° C. in a bag sealed to oxygen.

The frozen composition must be warmed to room temperature until a liquid form is found before use.

### Example 5: Useful Composition According to the Invention in Solid Form

An example of a useful composition according to the invention in freeze-dried form can be obtained by lyophilization of the composition of Example 4 in the frozen state.

### TEST RESULTS DEMONSTRATING THE EFFECT OF THE INVENTION

### In Vivo Demonstration of the Effect on the Prevention and/or Treatment of Hypertriglyceridemia

The objective of this study is to evaluate the effects of bacteria of the Christensenellaceae family on hypertriglyceridemia. The study was blinded: the experimenters did not know the treatments, so that their previous knowledge did not influence the result of the study.

The operating protocol is described below.

Four-week-old male and female C57BI/6 mice were purchased at Charles River (St Germain sur I′Arbresle, France) and received in the farm to begin a one-week acclimation period to the standard maintenance diet (Safe A04). On the first day of the experiment (D0), the animals were put on a high-fat diet (“Research Diet D12451” 45% kcal, noted HFDF45% in Table 1 for “half-fat diet 45%”) and randomly distributed into 2 groups (n = 5 per group/sex), each receiving a 150 µL solution of Christensenella minuta (10⁹ CFU/mL) or a control solution consisting of the culture medium used for bacteria growth for 12 weeks.

The animals received food and water at will throughout the duration of the study and were kept in a room at a constant temperature (22.0 ± 2.0° C.) and humidity (40-50%) and on a 12 hour (8 a.m. 8 p.m.)/12 hr cycle of illumination/darkness.

At the end of the trial (D85), a final blood sample was taken on the animals (anesthetized with a mixture of ketamine/xylazine 80/10 mg/kg). The blood samples were collected in centrifuge tubes pre-filled with heparin sulfate (200 IU/mL blood). Plasma was separated by centrifugation (3500 rpm, 15 min at 4° C.) and triglycerides were measured.

The results are shown in Table 3.

TABLE 3

| | | |
|---|---|---|
| HFD45% + C. minuta | C3SM | 0.42 |
| | C3OG | 0.80 |
| | C3OD | 0.65 |
| | C6SM | 0.43 |
| | C6OG | 0.35 |
| | C2SM | 1.20 |
| HFD45% + control | C2OG | 0.52 |
| | C7SM | 0.73 |
| | C7OG | 0.63 |
| | C7OD | 0.54 |
| HFD45% + C. minuta | C9SM | 0.41 |
| | C9OG | 0.56 |
| | C15SM | 0.31 |
| | C15OG | 0.42 |
| | C15OD | 1.23 |
| HFD45% + control | C12SM | 0.34 |
| | C12OG | 1.94 |
| | C12OD | 1.12 |
| | C14SM | 0.84 |
| | C14OG | 1.24 |

These results show that the administration of a bacterium from the Christensenellaceae family prevents the increase of plasma triglycerides and can be used for the prevention or treatment of hypertriglyceridemia.

### In Vivo Demonstration of the Effect on Hepatic Lipid Metabolism

The objective of this study is to evaluate the effects of bacteria of the Christensenellaceae family on hepatic lipid metabolism. The study was blinded: the experimenters did not know the treatments, so that their previous knowledge did not influence the result of the study.

The operating protocol is described below.

Four-week-old male C57BI/6 mice were purchased at Charles River (St Germain sur I′Arbresle, France) and received in the farm to begin a one-week acclimation period to the standard maintenance diet (Safe A04). On the first day of the experiment (D0), the animals were randomly divided into 4 groups (n=10 per group). A control group remained on the standard maintenance diet (Safe A04), while the other 3 groups were placed on a high-fat diet (“Research Diet D12451” 45% kcal denoted HFDF45% in the figures). These 3 groups also received daily a 150 µL solution containing either the bacterium Christensenella minuta (10⁹ CFU/mL), or Orlistat at a dose of 20 mg/kg (Orlistat is a drug used to limit the absorption fats and is used here as a positive control) or a control solution consisting of the buffer used to preserve the bacteria for 7 weeks. The control group maintained on the standard diet also received the control solution containing the storage buffer daily for the 7 weeks of the experiment.

The animals received food and water at will throughout the duration of the study and were kept in a room at a constant temperature (22.0 ± 2.0° C.) and humidity (40-50%) and on a 12 hour (8 a.m. 8 p.m.)/12 hr cycle of illumination/darkness. At the end of the test (D53), the animals were euthanized and the whole livers removed, then immediately frozen in liquid nitrogen and stored at -80° C. until analysis.

The liver samples (about 100 mg) were then ground in 1 mL of a 5% NP40 solution in ultra pure H2O, and incubated for 5 min at 90° C. The samples were brought to room temperature to cool and incubated a second time at 90° C. for 5 min to allow the solubilization of the TG and FFA. The supernatants containing the TG and FFA were separated from the debris by centrifugation.

The quantification of TG (Sigma-Aldrich, MAK266) and FFA (Sigma-Aldrich, MAK044) was carried out on the supernatants according to the recommendations of the manufacturer of the dedicated kits by colorimetry. The concentration of TG and FFA was determined by measuring the OD at 570 nm (FluoStar Omega reader, BMG Labtech). Statistical analysis was performed with the Dunnett’s multiple comparisons test method, with the reference group HFD45%-Veh.

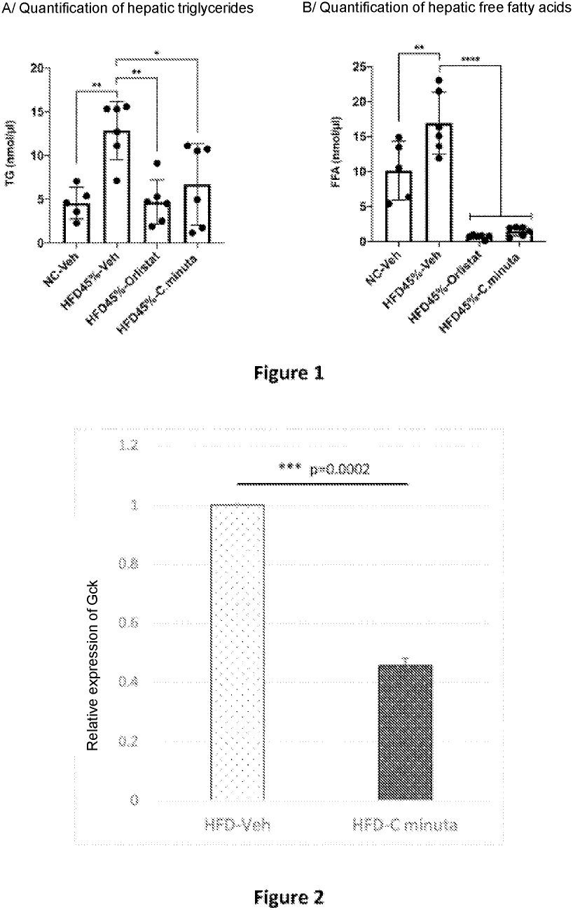

The results are shown in FIG. 1.

TG (FIG. 1A) and FFA (FIG. 1B) concentration was determined for mice on normal (NC) or high-fat diets under different oral treatment conditions (Vehicle (Veh), Orlistat, C. minuta).

As expected, quantification showed an increase in hepatic TG content in the control group on a high-fat diet (HFD45%-Veh) compared to the control group on a normal diet (NC-Veh) (p=0.0045). This increase is corrected by treating the animals with C. minuta (HFD45%-C. minuta, p=0.299) and Orlistat (HFD45%-Orlistat, reference molecule, p=0.0033), with a return to basal rate (NC-Veh).

Quantification of hepatic free fatty acids showed a slight increase in hepatic FFA content in the control group on a high-fat diet (HFD45%-Veh) compared to the control group on a normal diet (NC-Veh) (p=0.0176). Animals under HFD45% treated with C. minuta (HFD45%-C. minuta) and Orlistat (HFD45%-Orlistat, reference molecule) have a very low level of hepatic FFAs, which is lower than the control group HFD45%-Veh (p<0.0001), as well as than the NC-Veh control group (p<0.0001, for the 2 treatments, not shown in the figure).

### In Vivo Demonstration of the Effect on Energy Metabolism Regulation

The objective of this study is to evaluate the effects of bacteria from the family Christensenellaceae on the molecular signals involved in the regulation of energy metabolism at the hepatic level. The study was blinded: the experimenters did not know the treatments, so that their previous knowledge did not influence the result of the study.

The operating protocol is described below.

Four-week-old male C57BI/6 mice were purchased at Charles River (St Germain sur I′Arbresle, France) and received in the farm to begin a one-week acclimation period to the standard maintenance diet (Safe A04). On the first day of the experiment (D0), the animals were randomly divided into 4 groups (n=10 per group). A control group remained on the standard maintenance diet (Safe A04), while the other 3 groups were placed on a high-fat diet (“Research Diet D12451” 45% kcal denoted HFDF45% in the figures). These 3 groups also received daily a 150 µL solution containing either the bacterium Christensenella minuta (10⁹ CFU/mL), or Orlistat at a dose of 20 mg/kg (Orlistat is a drug used to limit the absorption fats and is used here as a positive control) or a control solution consisting of the buffer used to preserve the bacteria for 7 weeks. The control group maintained on the standard diet also received the control solution containing the storage buffer daily for the 4 weeks of the experiment.

The animals received food and water at will throughout the duration of the study and were kept in a room at a constant temperature (22.0 ± 2.0° C.) and humidity (40-50%) and on a 12 hour (8 a.m. 8 p.m.)/12 hr cycle of illumination/darkness.

At the end of the test (D27), the animals were euthanized and the whole livers removed, then immediately frozen in liquid nitrogen and stored at -80° C. until analysis.

The results are shown in FIG. 2.

Glucokinase is a hepatic enzyme catalyzing the first glucose phosphorylation reaction necessary for its entry into glycolysis in the form of glucose-6-phosphate. It is therefore a key enzyme in the regulation of this catabolic pathway leading to the production of acetyl-CoA, the founding metabolic element at the origin of lipid synthesis. Gck has been shown to be overexpressed in response to a high-fat diet in rodents [Tsukita, S., Yamada, T., Uno, K., Takahashi, K., Kaneko, K., Ishigaki, Y. et al. (2012). Hepatic Glucokinase Modulates Obesity Predisposition by Regulating BAT Thermogenesis via Neural Signals. Cell Metabolism, 16 (6), 825-832], and it has been shown that overexpression of hepatic Gck leads to hyperlipidemia [O′Doherty, R. M., Lehman, D. L., Télémaque-Potts, S., & Newgard, C. B. (1999). Metabolic impact of glucokinase overexpression in liver: lowering of blood glucose in fed rats is accompanied by hyperlipidemia. Diabetes, 48 (10), 2022-2027].

Thus, we can deduce that treatment with C. minuta (FIG. 2) inhibits the expression of the Gck gene, which leads to an inhibition of the entry of glucose into glycolysis, which reduces the influx of acetyl-CoA and limits the de novo synthesis of fatty acids in the liver intended to be stored as triglycerides before being released into the bloodstream. Through this action, the C. minuta treatment significantly reduces the quantity of triglycerides produced by the liver and has a hypolipidemic action useful in the treatment of hypertriglyceridemia. Thus, C. minuta reduces hepatic lipogenesis through inhibition of Gck expression.

1. A bacterium of the family Christensenellaceae for use in the prevention and/or treatment of hypertriglyceridemia in humans or animals.

2. The bacterium of the family Christensenellaceae for its use according to claim 1, characterized in that said bacterium is a bacterium of the genus Christensenella.

3. The bacterium of the family Christensenellaceae for use according to claim 1, characterized in that said bacterium is selected from Christensenella massiliensis, Christensenella timonensis and Christensenella minuta.

4. The bacterium of the family Christensenellaceae for use according to claim 1 in the prevention and/or treatment of secondary hypertriglyceridemia in humans or animals.

5. The bacterium of the family Christensenellaceae for use according to claim 1 in the prevention and/or treatment of primary hypertriglyceridemia in humans or animals.

6. A composition comprising at least one bacterium of the Christensenellaceae family for use according to claim 1, for the prevention and/or treatment of hypertriglyceridemia in humans or animals.

7. The composition for its use according to claim 6, characterized in that it is in liquid form.

8. The composition for its use according to claim 6, characterized in that it is in solid form.

9. The composition for its use according to claim 8, characterized in that the bacteria are present in freeze-dried form.

10. The composition for use according to claim 6, characterized in that the bacteria present are at least 50% living bacteria (by number).

11. The composition for use according to claim 6, characterized in that the bacteria present are at least 90% living bacteria (by number).

12. The composition for its use according to claim 6, orally, rectally, inhaled or topically.

13. The composition for its use according to claim 6, characterized in that it is in the form of powder, microencapsulated powder, gelcap, capsule, tablet, lozenge, granules, emulsion, suspension or suppository.

14. The composition for its use according to claim 6, characterized in that it is in a gastro-resistant form.

15. The composition for its use according to claim 6, characterized in that it also comprises:
at least one probiotic, and/or
at least one bacterium producing lactic acid and/or at least one other organism promoting the anaerobic conditions necessary for the survival of Christensenellaceae, and/or
at least one bacterium associated with the Christensenellaceae ecosystem, and/or
at least one bacterium chosen from bacteria of the genus Faecalibacterium, Akkermansia, Eubacterium, Turicibacter and Oscillospira, and/or
at least one prebiotic, and/or
at least one polyphenol, and/or
at least one mineral and/or at least one vitamin and/or at least one nutritional agent, and/or
at least one pharmaceutical active ingredient chosen from statins, fibrates, niacin, omega 3.
